SEQUENCE LISTING (105 sequences)

SEQ ID NO: 1 - protein, 486 aa, unknown
  Ply2638 A endolysin
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 2 - DNA, 1458 bp, unknown
  Ply2638A endolysin
atgctgaccgcaattgattacctgaccaaaaaaggctggaaaatctcttccgatccgcgtacctatgatggctatccgaaaaactacggctaccgcaactaccacgagaacggtatcaactatgatgaattttgcggcggctatcaccgtgcctttgatgtttactccaacgaaacgaacgatgtgccagcagttactagcggtactgttatcgaggcgaacgattacggtaacttcggcggtactttcgttatccgcgacgcgaacgacaacgattggatctatggccacctgcagcgcggttccatgcgttttgttgttggcgacaaagtgaaccagggcgacattattggtctgcagggtaacagcaactattacgacaacccgatgagcgttcacctgcatctgcagctgcgtccgaaagacgccaaaaaagacgaaaaaagccaggtttgctctggtctggcgatggagaaatacgatatcaccaacctgaacgcgaaacaggacaaaagcaaaaacggctctgtcaaagaactgaaacacatctactctaaccacattaaaggtaacaaaatcaccgctccgaaaccgtctattcagggtgtcgtgatccacaacgattacggttccatgactccaagccagtacctcccgtggctgtacgctcgtgaaaacaacggtactcacgttaacggctgggcatctgtatacgccaaccgcaacgaagtactgtggtaccacccgactgattatgttgaatggcattgcggtaaccagtgggcaaacgcgaacctgattggtttcgaagtctgtgaatcttacccgggtcgtatttctgacaaactgttcctggagaacgaagaggctactctcaaagtggctgcagacgtgatgaaaagctacggtctgccagtaaaccgtaacaccgttcgcctgcataacgaatttttcggcacctcctgtccgcaccgttcttgggatctgcacgttggtaaaggcgaaccgtacaccactacgaacatcaacaaaatgaaagactacttcatcaaacgtatcaaacactactatgatggcggcaaactggaagtatctaaagctgcgacgatcaaacagtccgacgtcaaacaggaggtaaaaaaacaggaggcgaaacagattgttaaagccaccgactggaaacagaacaaagacggcatctggtacaaagctgaacacgcttctttcaccgtgactgcaccagagggcattatcacgcgttataaaggtccgtggaccggtcatccacaggctggtgtgctgcagaaaggtcagacgatcaaatatgacgaggttcagaaattcgacggtcatgtatgggtatcttgggaaacgtttgaaggtgaaacggtctatatgccggtgcgcacttgggatgcgaaaaccggtaaagtgggcaaactgtggggtgaaatcaaa

SEQ ID NO: 3 - protein, 68 aa, unknown
  peptidoglycan binding domain of Ply2638A endolysin
YKAEHASFTVTAPEGIITRYKGPWTGHPQAGVLQKGQTIKYDEVQKFDGHVWVSWETFEGETVYMPVR

SEQ ID NO: 4 - protein, 100 aa, unknown
  M23 peptidase domain of Ply2638A endolysin
YHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDE

SEQ ID NO: 5 - protein, 127 aa, unknown
  putative amidase domain of Ply2638A endolysin
IQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRS

SEQ ID NO: 6 - protein, 485 aa, unknown
  Ply2638A endolysin without Met
LTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 7 - protein, 497 aa, unknown
  PK-Ply2638A
AMGKRKKRKKRKLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 8 - DNA, 1491 bp, unknown
  PK-Ply2638A
gccatgggcaagcgtaagaaacgcaaaaaacgcaaactgaccgcaattgattacctgaccaaaaaaggctggaaaatctcttccgatccgcgtacctatgatggctatccgaaaaactacggctaccgcaactaccacgagaacggtatcaactatgatgaattttgcggcggctatcaccgtgcctttgatgtttactccaacgaaacgaacgatgtgccagcagttactagcggtactgttatcgaggcgaacgattacggtaacttcggcggtactttcgttatccgcgacgcgaacgacaacgattggatctatggccacctgcagcgcggttccatgcgttttgttgttggcgacaaagtgaaccagggcgacattattggtctgcagggtaacagcaactattacgacaacccgatgagcgttcacctgcatctgcagctgcgtccgaaagacgccaaaaaagacgaaaaaagccaggtttgctctggtctggcgatggagaaatacgatatcaccaacctgaacgcgaaacaggacaaaagcaaaaacggctctgtcaaagaactgaaacacatctactctaaccacattaaaggtaacaaaatcaccgctccgaaaccgtctattcagggtgtcgtgatccacaacgattacggttccatgactccaagccagtacctcccgtggctgtacgctcgtgaaaacaacggtactcacgttaacggctgggcatctgtatacgccaaccgcaacgaagtactgtggtaccacccgactgattatgttgaatggcattgcggtaaccagtgggcaaacgcgaacctgattggtttcgaagtctgtgaatcttacccgggtcgtatttctgacaaactgttcctggagaacgaagaggctactctcaaagtggctgcagacgtgatgaaaagctacggtctgccagtaaaccgtaacaccgttcgcctgcataacgaatttttcggcacctcctgtccgcaccgttcttgggatctgcacgttggtaaaggcgaaccgtacaccactacgaacatcaacaaaatgaaagactacttcatcaaacgtatcaaacactactatgatggcggcaaactggaagtatctaaagctgcgacgatcaaacagtccgacgtcaaacaggaggtaaaaaaacaggaggcgaaacagattgttaaagccaccgactggaaacagaacaaagacggcatctggtacaaagctgaacacgcttctttcaccgtgactgcaccagagggcattatcacgcgttataaaggtccgtggaccggtcatccacaggctggtgtgctgcagaaaggtcagacgatcaaatatgacgaggttcagaaattcgacggtcatgtatgggtatcttgggaaacgtttgaaggtgaaacggtctatatgccggtgcgcacttgggatgcgaaaaccggtaaagtgggcaaactgtggggtgaaatcaaa

SEQ ID NO: 9 - protein, 500 aa, unknown
  Ply2638A-PK
AMGLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKGSGKRKKRKKRK

SEQ ID NO: 10 - DNA, 1500 bp, unknown
  Ply2638A-PK
gccatgggcctgaccgcaattgattacctgaccaaaaaaggctggaaaatctcttccgatccgcgtacctatgatggctatccgaaaaactacggctaccgcaactaccacgagaacggtatcaactatgatgaattttgcggcggctatcaccgtgcctttgatgtttactccaacgaaacgaacgatgtgccagcagttactagcggtactgttatcgaggcgaacgattacggtaacttcggcggtactttcgttatccgcgacgcgaacgacaacgattggatctatggccacctgcagcgcggttccatgcgttttgttgttggcgacaaagtgaaccagggcgacattattggtctgcagggtaacagcaactattacgacaacccgatgagcgttcacctgcatctgcagctgcgtccgaaagacgccaaaaaagacgaaaaaagccaggtttgctctggtctggcgatggagaaatacgatatcaccaacctgaacgcgaaacaggacaaaagcaaaaacggctctgtcaaagaactgaaacacatctactctaaccacattaaaggtaacaaaatcaccgctccgaaaccgtctattcagggtgtcgtgatccacaacgattacggttccatgactccaagccagtacctcccgtggctgtacgctcgtgaaaacaacggtactcacgttaacggctgggcatctgtatacgccaaccgcaacgaagtactgtggtaccacccgactgattatgttgaatggcattgcggtaaccagtgggcaaacgcgaacctgattggtttcgaagtctgtgaatcttacccgggtcgtatttctgacaaactgttcctggagaacgaagaggctactctcaaagtggctgcagacgtgatgaaaagctacggtctgccagtaaaccgtaacaccgttcgcctgcataacgaatttttcggcacctcctgtccgcaccgttcttgggatctgcacgttggtaaaggcgaaccgtacaccactacgaacatcaacaaaatgaaagactacttcatcaaacgtatcaaacactactatgatggcggcaaactggaagtatctaaagctgcgacgatcaaacagtccgacgtcaaacaggaggtaaaaaaacaggaggcgaaacagattgttaaagccaccgactggaaacagaacaaagacggcatctggtacaaagctgaacacgcttctttcaccgtgactgcaccagagggcattatcacgcgttataaaggtccgtggaccggtcatccacaggctggtgtgctgcagaaaggtcagacgatcaaatatgacgaggttcagaaattcgacggtcatgtatgggtatcttgggaaacgtttgaaggtgaaacggtctatatgccggtgcgcacttgggatgcgaaaaccggtaaagtgggcaaactgtggggtgaaatcaaagggtcgggaaagcgtaagaaacgcaaaaaacgcaaa

SEQ ID NO: 11 - protein, 6 aa, unknown
  synthetic peptide
KRKKRK

SEQ ID NO: 12 - protein, 9 aa, unknown
  synthetic peptide
KRKKRKKRK

SEQ ID NO: 13 - protein, 9 aa, unknown
  synthetic peptide
RRRRRRRRR

SEQ ID NO: 14 - protein, 8 aa, unknown
  synthetic peptide
KKKKKKKK

SEQ ID NO: 15 - protein, 10 aa, unknown
  synthetic peptide
KRKKRKKRKK

SEQ ID NO: 16 - protein, 12 aa, unknown
  synthetic peptide
KRKKRKKRKKRK

SEQ ID NO: 17 - protein, 14 aa, unknown
  synthetic peptide
KRKKRKKRKKRKKR

SEQ ID NO: 18 - protein, 16 aa, unknown
  synthetic peptide
KKKKKKKKKKKKKKKK

SEQ ID NO: 19 - protein, 19 aa, unknown
  synthetic peptide
KRKKRKKRKKRKKRKKRKK

SEQ ID NO: 20 - protein, 19 aa, unknown
  synthetic peptide
RRRRRRRRRRRRRRRRRRR

SEQ ID NO: 21 - protein, 19 aa, unknown
  synthetic peptide
KKKKKKKKKKKKKKKKKKK

SEQ ID NO: 22 - protein, 20 aa, unknown
  synthetic peptide
KRKKRKKRKRSKRKKRKKRK

SEQ ID NO: 23 - protein, 21 aa, unknown
  synthetic peptide
KRKKRKKRKRSKRKKRKKRKK

SEQ ID NO: 24 - protein, 21 aa, unknown
  synthetic peptide
KRKKRKKRKKRKKRKKRKKRK

SEQ ID NO: 25 - protein, 22 aa, unknown
  synthetic peptide
KRKKRKKRKRGSGKRKKRKKRK

SEQ ID NO: 26 - protein, 24 aa, unknown
  synthetic peptide
KRKKRKKRKRGSGSGKRKKRKKRK

SEQ ID NO: 27 - protein, 25 aa, unknown
  synthetic peptide
KRKKRKKRKKRKKRKKRKKRKKRKK

SEQ ID NO: 28 - protein, 31 aa, unknown
  synthetic peptide
KRKKRKKRKRSKRKKRKKRKRSKRKKRKKRK

SEQ ID NO: 29 - protein, 38 aa, unknown
  synthetic peptide
KRKKRKKRKRGSGSGKRKKRKKRKGSGSGKRKKRKKRK

SEQ ID NO: 30 - protein, 39 aa, unknown
  synthetic peptide
KRKKRKKRKKRKKRKKRKKRKKRKKRKKRKKRKKRKKRK

SEQ ID NO: 31 - protein, 42 aa, unknown
  synthetic peptide
KRKKRKKRKRSKRKKRKKRKRSKRKKRKKRKRSKRKKRKKRK

SEQ ID NO: 32 - protein, 5 aa, unknown
  synthetic peptide
  misc_feature (3)..(3): wherein Xaa is any other amino acid residue than lysine, arginine and histidine
KRXKR

SEQ ID NO: 33 - protein, 5 aa, unknown
  synthetic peptide
KRSKR

SEQ ID NO: 34 - protein, 5 aa, unknown
  synthetic peptide
KRGSG

SEQ ID NO: 35 - DNA, 36 bp, unknown
  Ply2638A forward primer
catgccatgggcctgaccgcaattgattacctgacc

SEQ ID NO: 36 - DNA, 40 bp, unknown
  Ply2638A reverse primer
ccgctcgagtcatcatttgatttcaccccacagtttgccc

SEQ ID NO: 37 - DNA, 63 bp, unknown
  PK-Ply2638A forward primer
catgccatgggcaagcgtaagaaacgcaaaaaacgcaaactgaccgcaattgattacctgacc

SEQ ID NO: 38 - DNA, 40 bp, unknown; identical to SEQ ID NO: 36
  PK-Ply2638A reverse primer

SEQ ID NO: 39 - DNA, 36 bp, unknown; identical to SEQ ID NO: 35
  Ply2638A-PK forward primer

SEQ ID NO: 40 - DNA, 74 bp, unknown
  Ply2638A-PK reverse primer
ccgctcgagtcatcatttgcgttttttgcgtttcttacgctttcccgaccctttgatttcaccccacagtttgc

SEQ ID NO: 41 - protein, 24 aa, unknown
  Pseudin 1
GLNTLKKVFQGLHEAIKLINNHVQ

SEQ ID NO: 42 - protein, 37 aa, unknown
  LL-37
LLGDFFRKSKEKIGKEFKRIVQRIKDFLRNLVPRTES

SEQ ID NO: 43 - protein, 13 aa, unknown
  Indolicidin
ILPWKWPWWPWRR

SEQ ID NO: 44 - protein, 23 aa, unknown
  Magainin
GIGKFLHSAKKFGKAFVGEIMNS

SEQ ID NO: 45 - protein, 25 aa, unknown
  Pleurocidin
GWGSFFKKAAHVGKHVGKAALTHYL

SEQ ID NO: 46 - protein, 36 aa, unknown
  Cecropin A (A.aegypti)
GGLKKLGKKLEGAGKRVFNAAEKALPVVAGAKALRK

SEQ ID NO: 47 - protein, 21 aa, unknown
  Buforin II
TRSSRAGLQFPVGRVHRLLRK

SEQ ID NO: 48 - protein, 39 aa, unknown
  Sarcotoxin IA
GWLKKIGKKIERVGQHTRDATIQGLGIAQQAANVAATAR

SEQ ID NO: 49 - protein, 29 aa, unknown
  SMAP-29
RGLRRLGRKIAHGVKKYGPTVLRIIRIAG

SEQ ID NO: 50 - protein, 54 aa, unknown
  OR-7
KTYYGTNGVHCTKNSLWGKVRLKNMKYDQNTTYMGRLQDILLGWATGAFGKTFH

SEQ ID NO: 51 - protein, 18 aa, unknown
  Protegrin
RGGRLCYCRRRFCVCVGR

SEQ ID NO: 52 - protein, 31 aa, unknown
  Cecropin P1
SWLSKTAKKLENSAKKRISEGIAIAIQGGPR

SEQ ID NO: 53 - protein, 40 aa, unknown
  Cecropin A (D. melanogaster)
GWLKKIGKKIERVGQHTRDATIQGLGIPQQAANVAATARG

SEQ ID NO: 54 - protein, 24 aa, unknown
  Ascaphine
GIKDWIKGAAKKLIKTVASHIANQ

SEQ ID NO: 55 - protein, 17 aa, unknown
  Apidaecine
ANRPVYIPPPRPPHPRL

SEQ ID NO: 56 - protein, 22 aa, unknown
  Nigrocine
GLLSKVLGVGKKVLCGVSGLVC

SEQ ID NO: 57 - protein, 19 aa, unknown
  Parasin 1
KGRGKQGGKVRAKAKTRSS

SEQ ID NO: 58 - protein, 25 aa, unknown
  Lycotoxin
IWLTALKFLGKHAAKKLAKQQLSKL

SEQ ID NO: 59 - protein, 18 aa, unknown
  Ranalexin
FLGGLIVPAMICAVTKKC

SEQ ID NO: 60 - protein, 26 aa, unknown
  Melittin
GIGAVLKVLTTGLPALISWIKRKRQQ

SEQ ID NO: 61 - protein, 38 aa, unknown
  Buforin I
GRGKQGGKVRAKAKTRSSRAGLQFPVGRVHRLLRKGNY

SEQ ID NO: 62 - protein, 13 aa, unknown
  Alpha 4
PNRAKRVITTFRT

SEQ ID NO: 63 - protein, 18 aa, unknown
  Artilysin1
GFFIPAVILPSIAFLIVP

SEQ ID NO: 64 - protein, 25 aa, unknown
  Artilysin2
GKPGWLIKKALVFKKLIRRPLKRLA

SEQ ID NO: 65 - protein, 27 aa, unknown
  WLBU2 variant
KRWVKRVKRVKRWVKRVVRVVKRWVKR

SEQ ID NO: 66 - protein, 526 aa, artificial
  LL-37-Ply2638A
MLLGDFFRKSKEKIGKEFKRIVQRIKDFLRNLVPRTESGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 67 - DNA, 1581 bp, artificial
  LL-37-Ply2638A
atgctcctgggtgacttctttcgcaaatccaaagagaaaatcggcaaagagttcaaacgtatcgtgcagcgcattaaagactttctgcgtaacctggttccgcgtaccgaatctggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 68 - protein, 518 aa, artificial
  SMAP-29-Ply2638A
MRGLRRLGRKIAHGVKKYGPTVLRIIRIAGGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 69 - DNA, 1557 bp, artificial
  SMAP-29-Ply2638A
atgcgtggtctgcgtcgcctgggtcgcaaaattgcgcacggcgtcaaaaaatacggcccgaccgtgctgcgcattatccgcatcgctggtggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 70 - protein, 525 aa, artificial
  Cecropin A (A.aegypti)-Ply2638A
MGGLKKLGKKLEGAGKRVFNAAEKALPVVAGAKALRKGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 71 - DNA, 1578 bp, artificial
  Cecropin A (A.aegypti)-Ply2638A
atgggcggcctgaaaaaactgggcaaaaaactggaaggtgccggcaaacgtgtgttcaacgctgcagaaaaagcactgccggttgtagctggtgctaaagctctccgtaaaggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 72 - protein, 528 aa, artificial
  Sarcotoxin IA-Ply2638A
MGWLKKIGKKIERVGQHTRDATIQGLGIAQQAANVAATARGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 73 - DNA, 1587 bp, artificial
  Sarcotoxin IA-Ply2638A
atgggatggctcaaaaagattggcaagaaaatcgagcgagtcggtcagcatacgcgtgatgcaactatccagggtttaggtatcgcacagcaagcagctaatgtagcagctactgctcggggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 74 - protein, 543 aa, artificial
  OR-7-Ply2638A
MKTYYGTNGVHCTKNSLWGKVRLKNMKYDQNTTYMGRLQDILLGWATGAFGKTFHGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 75 - DNA, 1632 bp, artificial
  OR-7-Ply2638A
atgaaaacctactatggcaccaacggcgtacactgcaccaaaaacagcctgtggggtaaagtgcgtctgaaaaacatgaaatacgaccagaacaccacgtatatgggccgtctccaggatatcctgctgggttgggcaactggtgccttcggcaaaacctttcacggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 76 - protein, 502 aa, artificial
  Indolicidin-Ply2638A
MILPWKWPWWPWRRGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 77 - DNA, 1509 bp, artificial
  Indolicidin-Ply2638A
atgatcctgccgtggaaatggccgtggtggccatggcgtcgcggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 78 - protein, 513 aa, artificial
  Pseudin1-Ply2638A
MGLNTLKKVFQGLHEAIKLINNHVQGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 79 - DNA, 1542 bp, artificial
  Pseudin1-Ply2638A
atgggtctgaacaccctgaaaaaagtattccagggtctgcatgaagcgattaaactgatcaacaaccacgttcagggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 80 - protein, 512 aa, artificial
  Magainin-Ply2638A
MGIGKFLHSAKKFGKAFVGEIMNSGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 81 - DNA, 1539 bp, artificial
  Magainin-Ply2638A
atgggtatcggcaaattcctgcactccgcaaaaaaattcggcaaagctttcgtgggcgaaattatgaactctggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 82 - protein, 514 aa, artificial
  Pleurocidin-Ply2638A
MGWGSFFKKAAHVGKHVGKAALTHYLGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 83 - DNA, 1545 bp, artificial
  Pleurocidin-Ply2638A
atgggctggggttctttctttaaaaaagcggctcacgttggcaaacatgtaggtaaagcagctctgacccactatctgggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 84 - protein, 510 aa, artificial
  BuforinII-Ply2638A
MTRSSRAGLQFPVGRVHRLLRKGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 85 - DNA, 1533 bp, artificial
  BuforinII-Ply2638A
atgacccgtagctctcgtgctggcctgcagtttccggttggtcgcgtgcaccgtctgctccgcaaaggatccatgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatga

SEQ ID NO: 86 - protein, 524 aa, artificial
  Ply2638A-LL-37
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSLLGDFFRKSKEKIGKEFKRIVQRIKDFLRNLVPRTES

SEQ ID NO: 87 - DNA, 1575 bp, artificial
  Ply2638A-LL-37
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccctcctgggtgacttctttcgcaaatccaaagagaaaatcggcaaagagttcaaacgtatcgtgcagcgcattaaagactttctgcgtaacctggttccgcgtaccgaatcttaa

SEQ ID NO: 88 - protein, 516 aa, artificial
  Ply2638A-SMAP-29
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSRGLRRLGRKIAHGVKKYGPTVLRIIRIAG

SEQ ID NO: 89 - DNA, 1551 bp, artificial
  Ply2638A-SMAP-29
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatcccgtggtctgcgtcgcctgggtcgcaaaattgcgcacggcgtcaaaaaatacggcccgaccgtgctgcgcattatccgcatcgctggttaa

SEQ ID NO: 90 - protein, 523 aa, artificial
  Ply2638A-Cecropin A (A.aegypti)
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSGGLKKLGKKLEGAGKRVFNAAEKALPVVAGAKALRK

SEQ ID NO: 91 - DNA, 1572 bp, artificial
  Ply2638A-Cecropin A (A.aegypti)
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccggcggcctgaaaaaactgggcaaaaaactggaaggtgccggcaaacgtgtgttcaacgctgcagaaaaagcactgccggttgtagctggtgctaaagctctccgtaaataa

SEQ ID NO: 92 - protein, 526 aa, artificial
  Ply2638A-Sarcotoxin IA
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSGWLKKIGKKIERVGQHTRDATIQGLGIAQQAANVAATAR

SEQ ID NO: 93 - DNA, 1581 bp, artificial
  Ply2638A-Sarcotoxin IA
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccggatggctcaaaaagattggcaagaaaatcgagcgagtcggtcagcatacgcgtgatgcaactatccagggtttaggtatcgcacagcaagcagctaatgtagcagctactgctcggtaa

SEQ ID NO: 94 - protein, 541 aa, artificial
  Ply2638A-OR-7
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSKTYYGTNGVHCTKNSLWGKVRLKNMKYDQNTTYMGRLQDILLGWATGAFGKTFH

SEQ ID NO: 95 - DNA, 1626 bp, artificial
  Ply2638A-OR-7
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccaaaacctactatggcaccaacggcgtacactgcaccaaaaacagcctgtggggtaaagtgcgtctgaaaaacatgaaatacgaccagaacaccacgtatatgggccgtctccaggatatcctgctgggttgggcaactggtgccttcggcaaaacctttcactaa

SEQ ID NO: 96 - protein, 500 aa, artificial
  Ply2638A-Indolicidin
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSILPWKWPWWPWRR

SEQ ID NO: 97 - DNA, 1503 bp, artificial
  Ply2638A-Indolicidin
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccatcctgccgtggaaatggccgtggtggccatggcgtcgctaa

SEQ ID NO: 98 - protein, 511 aa, artificial
  Ply2638A-Pseudin1
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSGLNTLKKVFQGLHEAIKLINNHVQ

SEQ ID NO: 99 - DNA, 1536 bp, artificial
  Ply2638A-Pseudin1
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccggtctgaacaccctgaaaaaagtattccagggtctgcatgaagcgattaaactgatcaacaaccacgttcagtaa

SEQ ID NO: 100 - protein, 510 aa, artificial
  Ply2638A-Magainin
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSGIGKFLHSAKKFGKAFVGEIMNS

SEQ ID NO: 101 - DNA, 1533 bp, artificial
  Ply2638A-Magainin
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccggtatcggcaaattcctgcactccgcaaaaaaattcggcaaagctttcgtgggcgaaattatgaactcttaa

SEQ ID NO: 102 - protein, 512 aa, artificial
  Ply2638A-Pleurocidin
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSGWGSFFKKAAHVGKHVGKAALTHYL

SEQ ID NO: 103 - DNA, 1539 bp, artificial
  Ply2638A-Pleurocidin
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccggctggggttctttctttaaaaaagcggctcacgttggcaaacatgtaggtaaagcagctctgacccactatctgtaa

SEQ ID NO: 104 - protein, 508 aa, artificial
  Ply2638A-BuforinII
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIKSTRSSRAGLQFPVGRVHRLLRK

SEQ ID NO: 105 - DNA, 1527 bp, artificial
  Ply2638A-BuforinII
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaatccacccgtagctctcgtgctggcctgcagtttccggttggtcgcgtgcaccgtctgctccgcaaataa

(19) United States
(12) Patent Application Publication
(10) Pub. No.: US 2017/0312344 A1
(43) Pub. Date: Nov. 2, 2017

(54) ANTIMICROBIAL AGENTS

(71) Applicant: LYSANDO AG, Triesenberg (LI)

(72) Inventor: Stefan MILLER, Regensburg (DE)

(21) Appl. No.: 15/590,293

(22) Filed: May 9, 2017

Related U.S. Application Data
(63) Continuation of application No. 14/113,596, filed on Dec. 31, 2013, now abandoned.

(30) Foreign Application Priority Data
Apr. 27, 2011 (EP) 11163897.9

(51) Int. Cl. A61K 38/47; A61K 39/085; A01N 63/00; A61K 45/06; C12N 9/36; C07K 2/00

(52) U.S. Cl. CPC A61K 38/47; C12N 9/2462; C07K 2/00; A01N 63/00; A61K 45/06; A61K 39/085; C07K 2319/00

(57) ABSTRACT

The present invention relates to a fusion protein comprising an endolysin with an amino acid sequence according to SEQ ID NO: 1 and fragments and/or derivatives thereof and an additional cationic or polycationic peptide, an amphipatic peptide, a sushi peptide, a defensin, a hydrophobic peptide or an antimicrobial peptide fused to said endolysin, fragment and/or derivative at the N- and/or C-terminus. Moreover, the present invention relates to nucleic acid molecules encoding said fusion protein, vectors comprising said nucleic acid molecules and host cells comprising either said nucleic acid molecules or said vectors. In addition, the present invention relates to said fusion protein for use as a medicament, in particular for the treatment or prevention of staphylococcal infections, as diagnostic means, as cosmetic substance or as sanitizing agent. The present invention also relates to the use of said fusion protein for the treatment or prevention of staphylococcal contamination of foodstuff, of food processing equipment, of food processing plants, of feed for livestock animals, of surfaces coming into contact with foodstuff, of medical devices, of surfaces in hospitals and surgeries. Furthermore, the present invention relates to a pharmaceutical composition comprising said fusion protein.

This application is a continuation of application of U.S. Ser. No. 14/113,596, filed Oct. 24, 2013, which application is a national phase application under 35 U.S.C. §371 of International Application No. PCT/EP2012/057808, filed Apr. 27, 2012, which claims priority to European Application No. 11163897.9, filed Apr. 27, 2011. The entire text of each of the above referenced disclosures is specifically incorporated herein by reference.

The present invention relates to a fusion protein comprising an endolysin with an amino acid sequence according to SEQ ID NO: 1 and fragments and/or derivatives thereof and an additional cationic or polycationic peptide, an amphipatic peptide, a sushi peptide, a defensin, a hydrophobic peptide or an antimicrobial peptide fused to said endolysin, fragment and/or derivative at the N- and/or C-terminus. Moreover, the present invention relates to nucleic acid molecules encoding said fusion protein, vectors comprising said nucleic acid molecules and host cells comprising either said nucleic acid molecules or said vectors. In addition, the present invention relates to said fusion protein for use as a medicament, in particular for the treatment or prevention of staphylococcal infections, as diagnostic means, as cosmetic substance or as sanitizing agent. The present invention also relates to the use of said fusion protein for the treatment or prevention of staphylococcal contamination of foodstuff, of food processing equipment, of food processing plants, of feed for livestock animals, of surfaces coming into contact with foodstuff, of medical devices, of surfaces in hospitals and surgeries. Furthermore, the present invention relates to a pharmaceutical composition comprising said fusion protein.

The bacterium Staphylococcus aureus is a facultative anaerobic, Gram-positive coccus. It occurs almost everywhere in the nature, as well as on the skin and the upper respiratory tract of about 25 to 30% humans. Usually Staphylococcus aureus does not cause any symptoms. Due to more favourable conditions or a weak immune system, Staphylococcus aureus may spread and thus, causes in humans infections of the skin, like pyoderma, particularly folliculitis, furuncle, carbuncle, abscesses of the sweat glands and pemphigus, and like scaled skin syndrome, endocarditis, toxic shock syndrome and sepsis. In cases such bacteria are resistant against several important antibiotics (multi-resistant), it is hard to eliminate these bacteria and they become very dangerous after infection of third parties.

Multi-resistant Staphylococcus aureus are Staphylococcus aureus strains which are resistant against all commercially available β-lactam antibiotics, e.g. penicillin. But they are often resistant against more than one class of antibiotics, such as chinolone, tetracyclins, aminoglycosides, erythromycin and sulfonamides. The increasing rate of multi-resistant S. aureus strains represents a serious problem in the therapy of infections due to S. aureus. Thus, there is a need for antimicrobial agents against Staphylococcus aureus.

Endolysins are peptidoglycan hydrolases encoded by bacteriophages (or bacterial viruses). They are synthesized during late gene expression in the lytic cycle of phage multiplication and mediate the release of progeny virions from infected cells through degradation of the bacterial peptidoglycan. They are either β-(1,4)-glycosylases (lysozymes), transglycosylases, amidases or endopeptidases. Antimicrobial application of endolysins was already suggested in 1991 by Gasson (GB2243611). Although the killing capacity of endolysins has been known for a long time, the use of these enzymes as antibacterials was ignored due to the success and dominance of antibiotics. Only after the appearance of multiple antibiotic resistant bacteria this simple concept of combating human pathogens with endolysins received interest. A compelling need to develop totally new classes of antibacterial agents emerged and endolysins used as ‘enzybiotics’—a hybrid term of ‘enzymes’ and ‘antibiotics’—perfectly met this need. In 2001, Fischetti and coworkers demonstrated for the first time the therapeutic potential of bacteriophage C1 endolysin towards group A streptococci (Nelson et al., 2001). Since then many publications have established endolysins as an attractive and complementary alternative to control bacterial infections, particularly by Gram-positive bacteria. Subsequently different endolysins against other Gram-positive pathogens such as Streptococcus pneumoniae (Loeffler et al., 2001), Bacillus anthracis (Schuch et al., 2002), S. agalactiae (Cheng et al., 2005) and Staphylococcus aureus (Rashel et al, 2007) have proven their efficacy as enzybiotics. However, it is also known that endolysins can, under some conditions (e.g. high ionic strength), create stable protoplast, where the internal bacterial cell pressure is not sufficient to lead to a cell burst. Under these conditions the bacterial cell wall can regenerate and the bacteria will survive.

Thus, there is a need for new antimicrobial agents against Staphylococcal bacteria.

This object is solved by the subject matter defined in the claims.

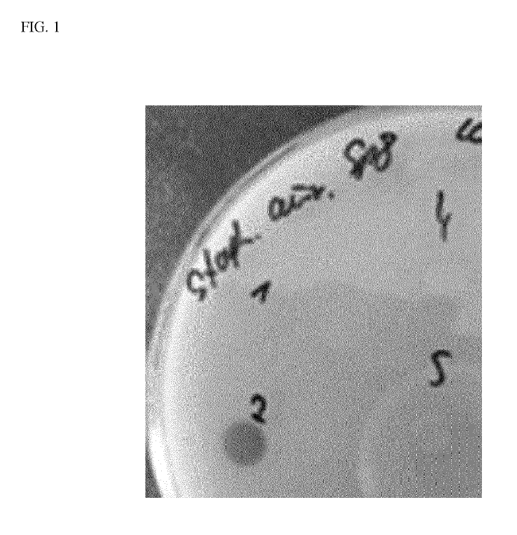

FIG. 1 shows a photograph of a Staph. aureus culture grown on an agar plate with a well-defined clear cut spot (black arrow; #2) that appeared within 16 h of incubation with Ply2638A-PK.

The term “protein” as used herein refers synonymously to the term “polypeptide”. The term “protein” as used herein refers to a linear polymer of amino acid residues linked by peptide bonds in a specific sequence. The amino-acid residues of a protein may be modified by e.g. covalent attachments of various groups such as carbohydrates and phosphate. Other substances may be more loosely associated with the polypeptide chains, such as heme or lipid, giving rise to the conjugated proteins which are also comprised by the term “protein” as used herein. There are various ways in which the polypeptide chains fold have been elucidated, in particular with regard to the presence of alpha helices and beta-pleated sheets. The term “protein” as used herein refers to all four classes of proteins being all-alpha, all-beta, alpha/beta and alpha plus beta. Moreover, the term “protein” refers to a complex, wherein the complex refers to a homomer.

The term “fusion protein” as used herein refers to an expression product resulting from the fusion of two or more nucleic acid sequences. Such a protein may be produced, e.g., in recombinant DNA expression systems. Moreover, the term “fusion protein” as used herein refers to a fusion of a first amino acid sequence, namely the endolysin Ply2638A and fragments and/or derivatives thereof, with a second or further amino acid sequences. The second or further amino acid sequence is preferably a peptide, in particular a cationic and/or a polycationic peptide, an amphipatic peptide, a sushi peptide, a defensin, a hydrophobic peptide and/or an antimicrobial peptide. Preferably, said second and/or further amino acid sequence is foreign to and not substantially homologous with any domain of the first amino acid sequence.

The term “peptide stretch” as used herein refers to any kind of peptide linked to a protein such as an endolysin. In particular the term “peptide stretch” as used herein refers to a cationic peptide or a polycationic peptide, an amphipatic peptide, a sushi peptide, a defensin, a hydrophobic peptide and/or an antimicrobial peptide. However, a peptide stretch in the meaning of the present invention does not refer to His-tags, preferably His₆₅-tags, His₆-tags, His₇-tags, His₈-tags, His₉-tags, His₁₀-tags, His₁₁-tags, His₁₂-tags, His₁₆-tags and His₂₀-tags, Strep-tags, Avi-tags, Myc-tags, Gst-tags, JS-tags, cystein-tags, FLAG-tags or other tags known in the art, thioredoxin or maltose binding proteins (MBP). The term “tag” in contrast to the term “peptide stretch” as used herein refers to a peptide which can be useful to facilitate expression and/or affinity purification of a polypeptide, to immobilize a polypeptide to a surface or to serve as a marker or a label moiety for detection of a polypeptide e.g. by antibody binding in different ELISA assay formats as long as the function making the tag useful for one of the above listed facilitation is not caused by the positively charge of said peptide. However, the His₆-tag may, depending on the respective pH, also be positively charged, but is used as affinity purification tool as it binds to immobilized divalent cations and is not used as a peptide stretch according to the present invention.

The term “peptide” as used herein refers to short polypeptides consisting of from about 2 to about 100 amino acid residues, more preferably from about 4 to about 50 amino acid residues, more preferably to about 5 to 30 amino acid residues, wherein the amino group of one amino acid residue is linked to the carboxyl group of another amino acid residue by a peptide bond. A peptide may have a specific function. A peptide can be a naturally occurring peptide or a synthetically designed and produced peptide. The peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). Preferred synthetically produced peptides are e.g. cationic, polycationic peptides, amphipatic peptides and hydrophobic peptides. Preferred naturally occurring peptides are e.g. amphipatic peptides, sushi peptides, defensines and antimicrobial peptides. The peptides have no or only low cell wall degrading activity but may have cell wall disrupting activity. A peptide in the meaning of the present invention does not refer to His-tags, Strep-tags, thioredoxin or maltose binding proteins (MBP) or the like, which are used to purify or locate proteins.

As used herein, the term “cationic peptide” refers to a peptide having positively charged amino acid residues. Preferably a cationic peptide has a pKa-value of 9.0 or greater. Typically, at least four of the amino acid residues of the cationic peptide can be positively charged, for example, lysine or arginine. “Positively charged” refers to the side chains of the amino acid residues which have a net positive charge at about physiological conditions. The term “cationic peptide” as used herein refers also to polycationic peptides.

The term “polycationic peptide” as used herein refers to a synthetically designed and produced peptide composed of mostly positively charged amino acid residues, in particular lysine, arginine and/or histidine residues, more preferably lysine and/or arginine residues. A peptide is composed of mostly positively charged amino acid residues if at least about 20, 30, 40, 50, 60, 70, 75, 80, 85, 90, 95 or about 100% of the amino acid residues are positively charged amino acid residues, in particular lysine and/or arginine residues. The amino acid residues being not positively charged amino acid residues can be neutrally charged amino acid residues and/or negatively charged amino acid residues and/or hydrophobic amino acid residues. Preferably the amino acid residues being not positively charged amino acid residues are neutrally charged amino acid residues, in particular serine and/or glycine.

The term, “antimicrobial peptide” (AMP) as used herein refers to any naturally occurring peptide that has microbicidal and/or microbistatic activity on for example bacteria, viruses, fungi, yeasts, mycoplasma and protozoa. Thus, the term “antimicrobial peptide” as used herein refers in particular to any peptide having anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, protozoicidal properties. The antimicrobial peptide may be a member of the RNAse A super family, a defensin, cathelicidin, granulysin, histatin, psoriasin, dermicidine or hepcidin. The antimicrobial peptide may be naturally occurring in insects, fish, plants, arachnids, vertebrates or mammals. Preferably the antimicrobial peptide may be naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human.

The term “sushi peptide” as used herein refers to complement control proteins (CCP) having short consensus repeats. The sushi module of sushi peptides functions as a protein-protein interaction domain in many different proteins. Peptides containing a Sushi domain have been shown to have antimicrobial activities. Preferably, sushi peptides are naturally occurring peptides.

The term “amphiphatic peptide” as used herein refers to synthetic peptides having both hydrophilic and hydrophobic functional groups. Preferably, the term “amphiphatic peptide” as used herein refers to a peptide having a defined arrangement of hydrophilic and hydrophobic groups e.g. amphiphatic peptides may be e.g. alpha helical, having predominantly non polar side chains along one side of the helix and polar residues along the remainder of its surface.

The term “hydrophobic group” as used herein refers to chemical groups such as amino acid side chains which are substantially water insoluble, but soluble in an oil phase, with the solubility in the oil phase being higher than that in water or in an aqueous phase. In water, amino acid residues having a hydrophobic side chain interact with one another to generate a nonaqueous environment. Examples of amino acid residues with hydrophobic side chains are valine, isoleucine, leucine, methionine, phenylalanine, tryptophan, cysteine, alanine, tyrosine, histidine, threonine, serine, proline and glycine residues.

The term “endolysin” as used herein refers to an enzyme which is suitable to hydrolyse bacterial cell walls. “Endolysins” comprise at least one “enzymatically active domain” (EAD) having at least one of the following activities: endopeptidase, N-acetyl-muramoyl-L-alanine-amidase (amidase), N-acetyl-muramidase, N-acetyl-glucosaminidase (lysozyme) or transglycosylases. In addition, the endolysins may contain also regions which are enzymatically inactive, and bind to the cell wall of the host bacteria, the so-called CBDs (cell wall binding domains). The endolysin may contain one, two or more CBDs. However, the term “endolysin” as used herein refers also to enzymes having at least one EAD but no CBDs. Generally, the cell wall binding domain is able to bind different components on the surface of bacteria. Preferably, the cell wall binding domain is a peptidoglycan binding domain and binds to the bacteria's peptidoglycan.

The term “cell wall” as used herein refers to all components that form the outer cell enclosure of the Gram-positive bacteria and thus guarantee their integrity. In particular, the term “cell wall” as used herein refers to peptidoglycan, the bacterial cell membrane, but also to additional layers deposited on the peptidoglycan as e.g. capsules, outer protein layers or slimes. The fusion proteins according to the present invention have a cell wall degrading activity, i.e. they cleave the components, e.g. the peptidoglycan enzymatically. In contrast the peptides do have no or only very low enzymatic activity but may have cell wall disrupting, i.e. physically disrupting activity.

The term “EAD” as used herein refers to the enzymatically active domain of an endolysin. The EAD is responsible for hydrolysing bacterial peptidoglycans. It exhibits at least one enzymatic activity of an endolysin. The EAD can also be composed of more than one enzymatically active module. The term “EAD” is used herein synonymously with the term “catalytic domain”.

The term “domain linker” as used herein refers to an amino acid sequence functioning to connect single protein domains with one another. As a rule domain linkers form no or only few regular secondary structure like α-helices or β-sheets and can occupy different conformations with the respective structural context. Methods to detect domain linker and properties of linker sequences are well known in the art as e.g. described in Bae et al., 2005, Bioinformatics, 21, 2264-2270 or George & Heringa, 2003, Protein Engineering, 15, 871-879. The term “deletion” as used herein refers to the removal of 1, 2, 3, 4, 5 or more amino acid residues from the respective starting sequence.

The term “insertion” or “addition” as used herein refers to the insertion or addition of 1, 2, 3, 4, 5 or more amino acid residues to the respective starting sequence.

The term “substitution” as used herein refers to the exchange of an amino acid residue located at a certain position for a different one.

The present invention relates to new antibacterial agents against staphylococcal bacteria. Surprisingly the inventors found that in comparison to the wild type Ply2638A endolysin that has only very low bacterial cell wall degrading, i.e. antibacterial activity on Staphylococcus aureus strains the fusion proteins according to the present invention have a very strong antibacterial activity on Staphylococcus aureus strains. Thus, one aspect of the present invention relates to a fusion protein comprising the endolysin Ply2638A comprising an amino acid sequence according to SEQ ID NO: 1, a fragment and/or derivative thereof and an additional cationic, polycationic peptide, an amphipatic peptide, a sushi peptide, a defensin, a hydrophobic peptide and/or an antimicrobial peptide fused to the N- and/or C-terminus of said endolysin, fragment and/or derivative thereof. The endolysin Ply2638 comprising an amino acid sequence according to SEQ ID NO: 1 is preferably encoded by a nucleotide sequence according to SEQ ID NO: 2.

The endolysin Ply2638 (GENBANK Accession number YP_239818) having an amino acid sequence according to SEQ ID NO: 1 has a length of 486 amino acids. It comprises a C-terminal cell wall binding domain (CBD) and two N-terminal enzymatic active domains (EADs). The putative C-terminal CBD is a peptidoglycan binding domain (PGB, aa 401-468) having an amino acid sequence according to SEQ ID NO: 3. The N-terminal EADs consists of a putative M23 peptidase (aa 47-146) according to SEQ ID NO: 4 and a putative amidase domain (aa 199-325) according to SEQ ID NO: 5. The PGB and the catalytic domains of the endolysin Ply2638 are connected by linker sequences.

Preferred fragments of the endolysin Ply2638A according to the present invention are polypeptides comprising an amino acid sequence according to SEQ ID NO: 3, according to SEQ ID NO: 4 and/or according to SEQ ID NO: 5. Another preferred fragment of the endolysin according to the present invention comprises an amino acid sequence according to SEQ ID NO: 6. The fragment having an amino acid sequence according to SEQ ID NO: 6 differs from the endolysin having an amino acid sequence according to SEQ ID NO: 1 in that the starting methionine residue has been deleted.

The derivatives of the endolysin according to the present invention are polypeptides comprising an amino acid sequence according to SEQ ID NO: 1, 3, 4, 5 and/or 6 but having additional modification and/or alterations. Said modifications and/or alterations can be mutations in particular deletions, insertions, additions, substitutions or any combinations thereof and/or chemical changes of the amino acid residues, e.g. biotinylation, acetylation, pegylation, chemical changes of the amino-, SH- or carboxyl-groups. Said derivatives according to the present invention exhibit the lytic activity of the Ply2638 endolysin or the activity of the fragments according to the present invention. Said activity can be about 10, 20, 30, 40, 50, 60, 70, 80, 90, 100, 110, 120, 130, 140, 150, 160, 170, 180, 190 or about 200% of the activity of the Ply2638A and/or the activity of the fragments according to the present invention. The activity can be measured by assays well known in the art by a person skilled in the art as e.g. the plate lysis assay or the liquid lysis assay which are e.g. described in Briers et al., J. Biochem. Biophys Methods 70: 531-533, (2007) or similar publications.

In a preferred embodiment of the present invention the fusion protein comprising the endolysin according to SEQ ID NO: 1, a fragment and/or derivative thereof and an additional cationic or polycationic peptide, an amphipatic peptide, a sushi peptide, a defensin, a hydrophobic peptide and/or an antimicrobial peptide at the N- and/or C-terminus of said endolysin, fragment and/or derivative thereof according to the present invention comprises additionally a tag such as a His₆-tag, Strep-tag, Avi-tag, Myc-tag, Gst-tag, JS-tag, cystein-tag, FLAG-tag or other tags known in the art at the N-terminus and/or at the C-terminus. In a preferred embodiment of the present invention said tag is linked to the fusion protein according to the present invention at the C-terminus. Said tag may be linked to said fusion protein over additional amino acid residues. Said additional amino acid residues may be at least 1, 2, 3, 4, 5, 6, 7, 8, 9 or 10 additional amino acid residues. In a preferred embodiment of the present invention the tag is linked to the fusion protein according to the present invention by the additional amino acid residues Leu-Glu or Lys-Gly.

The peptide according to the present invention is preferably covalently bound to the endolysin, fragment and/or derivative thereof according to the present invention. Preferably, said peptide consists of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acid residues. Especially preferred is a peptide comprising about 5 to about 100 amino acid residues, about 5 to about 50 or about 5 to about 30 amino acid residues. More preferred is a peptide comprising about 6 to about 42 amino acid residues, about 6 to about 39 amino acid residues, about 6 to about 38 amino acid residues, about 6 to about 31 amino acid residues, about 6 to about 25 amino acid residues, about 6 to about 24 amino acid residues, about 6 to about 22 amino acid residues, about 6 to about 21 amino acid residues, about 6 to about 20 amino acid residues, about 6 to about 19 amino acid residues, about 6 to about 16 amino acid residues, about 6 to about 14 amino acid residues, about 6 to about 12 amino acid residues, about 6 to about 10 amino acid residues or about 6 to about 9 amino acid residues.

Preferably, the peptide is no tag such as a His₆-tag, Strep-tag, Avi-tag, Myc-tag, Gst-tag, JS-tag, cystein-tag, FLAG-tag or other tags known in the art and no thioredoxin or maltose binding proteins (MBP). However, the peptide may comprise in addition such tag or tags or the like, which are used to purify or locate proteins.

Preferably, the peptide has the function to facilitate lysis of Gram-positive bacteria by the fusion protein according to the present invention but has no or only low cell wall degrading activity when administered without being fused to the endolysin, fragment and/or derivative thereof according to the present invention. Subsequently, the antibacterial activity of the fusion protein consisting of the endolysin, fragment and/or derivative thereof according to the present invention and the peptide to be tested can be compared to the endolysin, fragment and/or derivative thereof according the present invention having no peptide as also described in the Examples of the present invention. Preferably, said tests may be carried out on staphylococcal cells as used in the Examples of the present invention. In case the fusion protein has an increased antibacterial activity in comparison to the endolysin, fragment and/or derivative thereof according to the present invention without said peptide for at least one of the tested staphylococcal bacteria species then said peptide has a cell wall disrupting activity. Preferably, the antibacterial activity (in logarithmic units (=log₁₀ N₀/Nᵢ)) of the endolysin, fragment and/or derivative thereof according to the present invention is increased, preferably by at least about 5%, more preferably by at least about 10%, 20%, 30%, 40% 50% 60%, 70%, 80%, 90% or 100% or by any value in the range of up to 100% or even more by a peptide having cell wall disrupting activity.

In a preferred embodiment the peptide according to the present invention is selected from the group of cationic peptides and polycationic peptides.

In one aspect of the present invention the peptide is an cationic and/or polycationic peptide, which comprises one or more of the positively charged amino acid residues of lysine, arginine and/or histidine, in particular of lysine and/or arginine. Preferably, more than about 20, 30, 40, 50, 60, 70, 75, 80, 85, 90, 95 or 99% of the amino acid residues in said peptide are positively charged amino acid residues, in particular lysine and/or arginine residues. Especially preferred are peptides consisting of about 100% positively charged amino acid residues, in particular arginine and/or lysine residues, wherein preferably about 60% to about 70% of said positively charged amino acid residues are lysine residues and about 30% to about 40% of said positively charged amino acid residues are arginine residues. More preferred is a peptide consisting of about 100% positively charged amino acid residues, in particular arginine and/or lysine residues, wherein preferably about 64% to about 68% of said positively charged amino acid residues are lysine and about 32% to about 36% of said positively charged amino acid residues are arginine. Peptides consisting of either only arginine or only lysine are also preferred.

Especially preferred are cationic and/or polycationic peptides comprising at least one motive according to SEQ ID NO: 11 (KRKKRK). In particular cationic peptides comprising at least 2, 3, 4, 5, 6, 7, 8, 9, 10, 11, 12, 13, 14, 15, 16 or 17 motives according to SEQ ID NO: 11 (KRKKRK) are preferred. More preferred are cationic peptides comprising at least one KRK motive (lys-arg-lys), preferable at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 KRK motives.

In another preferred embodiment of the present invention the cationic peptide comprises beside the positively charged amino acid residues, in particular lysine and/or arginine residues, neutrally charged amino acid residues, in particular glycine and/or serine residues. Preferred are cationic peptides consisting of about 70% to about 100%, or about 80% to about 95%, or about 85% to about 90% positively charged amino acid residues, in particular lysine and/or arginine residues and of about 0% to about 30%, or about 5% to about 20%, or about 10% to about 20% neutrally charged amino acid residues, in particular glycine and/or serine residues. Preferred are peptides consisting of about 4% to about 8% serine residues, of about 33% to about 36% arginine residues and of about 56% to about 63% lysine residues. Especially preferred are peptides comprising at least one motive according to SEQ ID NO: 32 (KRXKR), wherein X is any other amino acid residue than lysine, arginine and histidine. Especially preferred are peptides comprising at least one motive according to SEQ ID NO: 33 (KRSKR). More preferred are cationic peptides comprising at least about 2, 3, 4, 5, 6, 7, 8, 9, 10, 11, 12, 13, 14, 15, 16, 17, 18, 19 or about 20 motives according to SEQ ID NO: 32 (KRXKR) or SEQ ID NO: 33 (KRSKR).

Also preferred are peptides consisting of about 9 to about 16% glycine residues, of about 4 to about 11 serine residues, of about 26 to about 32% arginine residues and of about 47 to about 55% lysine residues. Especially preferred are peptides comprising at least one motive according to SEQ ID NO: 34 (KRGSG). More preferred are cationic peptides comprising at least about 2, 3, 4, 5, 6, 7, 8, 9, 10, 11, 12, 13, 14, 15, 16, 17, 18, 19 or about 20 motives according to SEQ ID NO: 34 (KRGSG).

In another preferred embodiment of the present invention the cationic peptide comprises beside the positively charged amino acid residues, in particular lysine and/or arginine residues, hydrophobic amino acid residues, in particular valine, isoleucine, leucine, methionine, phenylalanine, tryptophan, cysteine, alanine, tyrosine, histidine, threonin, serine, proline and/or glycine residues. Preferred are cationic peptides consisting of about 70% to about 100%, or about 80% to about 95%, or about 85% to about 90% positively charged amino acid residues, in particular lysine and/or arginine residues and of about 0% to about 30%, or about 5% to about 20%, or about 10% to about 20% hydrophobic amino acid residues in particular valine, isoleucine, leucine, methionine, phenylalanine, tryptophan, cysteine, alanine, tyrosine, histidine, threonin, serine, proline and/or glycine residues.

Especially preferred peptides are presented in the following table:

TABLE 1

| Peptide | length | SEQ ID NO: |
|---|---|---|
| KRKKRK | 6 | SEQ ID NO: 11 |
| KRKKRKKRK | 9 | SEQ ID NO: 12 |
| RRRRRRRRR | 9 | SEQ ID NO: 13 |
| KKKKKKKK | 8 | SEQ ID NO: 14 |
| KRKKRKKRKK | 10 | SEQ ID NO: 15 |
| KRKKRKKRKKRK | 12 | SEQ ID NO: 16 |
| KRKKRKKRKKRKKR | 14 | SEQ ID NO: 17 |
| KKKKKKKKKKKKKKKK | 16 | SEQ ID NO: 18 |
| KRKKRKKRKKRKKRKKRKK | 19 | SEQ ID NO: 19 |
| RRRRRRRRRRRRRRRRRRR | 19 | SEQ ID NO: 20 |
| KKKKKKKKKKKKKKKKKKK | 19 | SEQ ID NO: 21 |
| KRKKRKKRKRSKRKKRKKRK | 20 | SEQ ID NO: 22 |
| KRKKRKKRKRSKRKKRKKRKK | 21 | SEQ ID NO: 23 |
| KRKKRKKRKKRKKRKKRKKRK | 21 | SEQ ID NO: 24 |
| KRKKRKKRKRGSGKRKKRKKRK | 22 | SEQ ID NO: 25 |
| KRKKRKKRKRGSGSGKRKKRKKRK | 24 | SEQ ID NO: 26 |
| KRKKRKKRKKRKKRKKRKKRKKRKK | 25 | SEQ ID NO: 27 |
| KRKKRKKRKRSKRKKRKKRKRSKRKKRKKRK | 31 | SEQ ID NO: 28 |
| KRKKRKKRKRGSGSGKRKKRKKRKGSGSGKRKKRKKRK | 38 | SEQ ID NO: 29 |
| KRKKRKKRKKRKKRKKRKKRKKRKKRKKRKKRKKRKKRK | 39 | SEQ ID NO: 30 |
| KRKKRKKRKRSKRKKRKKRKRSKRKKRKKRKRSKRKKRKKRK | 42 | SEQ ID NO: 31 |

In a further preferred embodiment of the present invention the fused peptide stretch is an amphipathic peptide, which comprises one or more of the positively charged amino acid residues of lysine, arginine and/or histidine, combined to one or more of the hydrophobic amino acid residues of valine, isoleucine, leucine, methionine, phenylalanine, tryptophan, cysteine, alanine, tyrosine, histidine, threonin, serine, proline and/or glycine. Side chains of the amino acid residues are preferably oriented in order that cationic and hydrophobic surfaces are clustered at opposite sides of the peptide. Preferably, more than about 30, 40, 50, 60 or 70% of the amino acids in said peptide are positively charged amino acids. Preferably, more than about 30, 40, 50, 60 or 70% of the amino acid residues in said peptide are hydrophobic amino acid residues. Advantageously, the amphipathic peptide is fused at the N-terminal and/or the C-terminal end of the polypeptide, fragment and/or derivative according to the present invention having cell wall degrading activity, thus enhancing the amphipathicity of the latter proteins.

In a preferred embodiment at least about 30, 40, 50, 60 or 70% of the said amino acid residues of the amphipathic peptide are either arginine or lysine residues and/or at least about 30, 40, 50, 60 or 70% of the said amino acid residues of the amphipathic peptide are of the hydrophobic amino acid residues valine, isoleucine, leucine, methionine, phenylalanine, tryptophan, cysteine, alanine, tyrosine, histidine, threonin, serine, proline and/or glycine.

Preferred amphipatic peptides are Pleurocidin according to SEQ ID NO:45, Cecropin P1 according to SEQ ID NO:52, Buforin II according to SEQ ID NO:47, Buforin I according to SEQ ID NO:61 and Magainin according to SEQ ID NO:44. Further preferred amphipatic peptides are Cathelidicine e.g. LL-37 according to SEQ ID NO:43.

In a further aspect of the present invention the fused peptide stretch is an antimicrobial peptide, which comprises a positive net charge and around 50% hydrophobic amino acid residues. The antimicrobial peptides are amphipathic, with a length of about 12 to about 50 amino acid residues.

Preferred antimicrobial peptides are listed in the following Table 2.

| Peptid | Sequenz | |
|---|---|---|
| LL-37 | LLGDFFRKSKEKIGKEFKRIVQRIKDFLRNLVPRTES | SEQ ID NO: 42 |
| SMAP-29 | RGLRRLGRKIAHGVKKYGPTVLRIIRIAG | SEQ ID NO: 49 |
| Indolicidin | ILPWKWPWWPWRR | SEQ ID NO: 43 |
| Protegrin | RGGRLCYCRRRFCVCVGR | SEQ ID NO: 51 |
| Cecropin P1 | SWLSKTAKKLENSAKKRISEGIAIAIQGGPR | SEQ ID NO: 52 |
| Magainin | GIGKFLHSAKKFGKAFVGEIMNS | SEQ ID NO: 44 |
| Pleurocidin | GWGSFFKKAAHVGKHVGKAALTHYL | SEQ ID NO: 45 |
| Cecropin A | GGLKKLGKKLEGAGKRVFNAAEKALPVVAGAKALRK | SEQ ID NO: 46 |
| (A.aegypti) | | |
| Cecropin A (D. | GWLKKIGKKIERVGQHTRDATIQGLGIPQQAANVAATARG | SEQ ID NO: 53 |
| melanogaster) | | |
| Buforin II | TRSSRAGLQFPVGRVHRLLRK | SEQ ID NO: 47 |
| Sarcotoxin IA | GWLKKIGKKIERVGQHTRDATIQGLGIAQQAANVAATAR | SEQ ID NO: 48 |
| Ascaphine | GIKDWIKGAAKKLIKTVASHIANQ | SEQ ID NO: 54 |
| Apidaecine | ANRPVYIPPPRPPHPRL | SEQ ID NO: 55 |
| Nigrocine | GLLSKVLGVGKKVLCGVSGLVC | SEQ ID NO: 56 |
| Pseudin 1 | GLNTLKKVFQGLHEAIKLINNHVQ | SEQ ID NO: 41 |
| Parasin 1 | KGRGKQGGKVRAKAKTRSS | SEQ ID NO: 57 |
| Lycotoxin | IWLTALKFLGKHAAKKLAKQQLSKL | SEQ ID NO: 58 |
| Ranalexin | FLGGLIVPAMICAVTKKC | SEQ ID NO: 59 |
| Melittin | GIGAVLKVLTTGLPALISWIKRKRQQ | SEQ ID NO: 60 |
| OR-7 | KTYYGTNGVHCTKNSLWGKVRLKNMKYDQNTTYM | SEQ ID NO: 50 |
| | GRLQDILLGWATGAFGKTFH | |
| Buforin I | AGRGKQGGKVRAKAKTRSSRAGLQFPVGRVHRLLRKGNY | SEQ ID NO: 61 |

In a further aspect of the present invention the fused peptide stretch is a sushi peptide which is described by Ding J L, Li P, Ho B Cell Mol Life Sci. 2008 April; 65(7-8):1202-19. The Sushi peptides: structural characterization and mode of action against Gram-negative bacteria.

Preferred sushi peptides are sushi peptides S1 and S3 and multiples thereof; FASEB J. 2000 September; 14(12):1801-13.

In a further aspect of the present invention the fused peptide stretch is a defensin, preferably Cathelicidine, Cecropin P1, Cecropin A or Magainin II.

In a further aspect of the present invention the fused peptide stretch is a hydrophobic peptide, preferably having the amino acid sequence Phe-Phe-Val-Ala-Pro.

Further preferred peptide stretches are listed in the following Table 3:

| | | |
|---|---|---|
| Alpha 4 | PNRAKRVITTFRT | SEQ ID |
| | | NO: 62 |
| Artilysin1 | GFFIPAVILPSIAFLIVP | SEQ ID |
| | | NO: 63 |
| Artilysin2 | GKPGWLIKKALVFKKLIRRPLKRLA | SEQ ID |
| | | NO: 64 |
| WLBU2 | KRWVKRVKRVKRWVKRVVRVVKRWVKR | SEQ ID |
| variant | | NO: 65 |

In another preferred embodiment of the present invention the peptide of the fusion protein according to the present invention comprise modifications and/or alterations of the amino acid sequences. Such alterations and/or modifications may comprise mutations such as deletions, insertions and additions, substitutions or combinations thereof and/or chemical changes of the amino acid residues, e.g. biotinylation, acetylation, peglyation, chemical changes of the amino-, SH- or carboxyl-groups.

A fusion protein according to the present invention as already outlined above is composed of
(a) an endolysin, fragment and/or derivative thereof according to the present invention, and
(b) a peptide fused to said endolysin, fragment and/or derivative thereof at the N- or C-Terminus, and optionally
(c) a tag, such as a His-tag, Strep-tag, Avi-tag, Myc-tag, Gst-tag, JS-tag, cystein-tag, FLAG-tag or other tags known in the art at the N- and/or C-Terminus.

The two and three components of the fusion protein, respectively, as outlined above may be linked to each other over additional amino acid residues e.g. due to cloning reasons. Moreover, the peptide may be linked to the N- and/or C-terminus of the endolysin, fragment and/or derivative thereof by additional amino acid residues. Said additional amino acid residues may be at least 1, 2, 3, 4, 5, 6, 7, 8, 9 or 10 additional amino acid residues. In a preferred embodiment of the present invention the peptide is linked to the endolysin, fragment and/or derivative according to the present invention by the additional amino acid residues Gly-Ser, Gly-Gly-Ser or Gly-Ser-Gly. The additional amino acid residues linking the N- and/or C-terminus of the endolysin, fragment and/or derivative thereof and the peptide are preferably Gly-Ser-Gly. In case the fusion protein additionally comprises a tag, the fusion protein according to the present invention is preferably linked to said tag by the additional amino acid residues Leu-Glu or Lys-Gly. Preferably the peptide comprises the amino acid methionine (Met), methionine, glycine and serine (Met-Gly-Ser) or alanine, methionine or glycine (Ala-Met-Gly).

Especially preferred is a fusion protein comprising an endolysin, fragment and/or derivative according to the present invention and a peptide having an amino acid sequence according to SEQ ID NO: 12 at the N- and/or C-terminus of the endolysin, fragment and/or derivative according to the present invention. More preferred are fusion proteins having an amino acid sequence according to SEQ ID NO: 7 and SEQ ID NO: 9. Also preferred are fusion proteins having an amino acid sequence according to SEQ ID NO: 7 and SEQ ID NO: 9 with an additional His₆-tag linked to the C- and/or N-terminus of the fusion protein. The fusion proteins having an amino acid sequence according to SEQ ID NO: 7 and SEQ ID NO: 9, respectively, with an additional C-terminal and/or N-terminal His₆-tag linked to the C- and/or N-terminus of the fusion protein by the additional amino acid residues lysine and glycine (Lys-Gly).

In case the peptide is fused to the endolysin, fragment and/or derivative thereof according to the present invention at the C-Terminus, the fusion protein comprises the additional tag preferably at the N-terminus. In an especially preferred embodiment of the present invention the peptide is fused to the endolysin, fragment and/or derivative thereof according to the present invention at the N-Terminus the fusion protein comprises an additional tag preferably at the C-terminus.

Fusion proteins are constructed by linking at least two nucleic acid sequences using standard cloning techniques as described e.g. by Sambrook et al. 2001, Molecular Cloning: A Laboratory Manual. Such a protein may be produced, e.g., in recombinant DNA expression systems. Such fusion proteins according to the present invention can be obtained by fusing the nucleic acids for endolysin, fragment and/or derivative thereof and the respective peptide.

As some fusion proteins may either be toxic upon expression in bacteria, or not homogenous due to protein degradation, the strategy might be to express these fusion proteins fused or linked to other additional proteins. Example for these other additional protein is Thioredoxin, which was shown to mediate expression of toxic antimicrobial peptides in E. coli (TrxA mediating fusion expression of antimicrobial peptide CM4 from multiple joined genes in Escherichia coli. Zhou L, Zhao Z, Li B, Cai Y, Zhang S. Protein Expr Purif. 2009 April; 64(2):225-230).

For antimicrobial function of the fusion proteins according to the present invention it may be necessary to remove the additional fusion protein by proteolytic cleavage. Commercially available kits like the pET32 expression system (Novagen), may need to modify e.g. the N-terminus of the fusion depending on the protease used, like from MGS to AMGS, were the remaining alanine residue results from an introduced Enterokinase cleavage site.

In a preferred embodiment of the present invention the fusion protein consists of a peptide according to SEQ ID NO: 11 to 34 and an endolysin according to SEQ ID NO: 1, 3, 4, 5 and/or 6. In a preferred embodiment the fusion protein comprises a peptide selected from the group of peptides according to SEQ ID NO: 11 to 34 and an endolysin selected from the group of endolysins according to SEQ ID NO: 1, 3, 4, 5 and/or 6.

The following table exemplifies the above outlined assembly of specifically preferred fusion proteins according to the present invention listed in the first column starting with the starting methionine residue at the N-terminus in the second column and ending with the optional tag at the C-terminus in the last column:

TABLE 4

| | | | | | polypeptide, | | |
|---|---|---|---|---|---|---|---|
| Fusion | | | | | fragment, | | |
| protein | | | | | derivative | | |
| according | | | | | according | | |
| to the | First | | | | to the | | |
| present | amino | | peptide | | present | | |
| invention | acid | additional | stretch | additional | invention | additional | |
| (SEQ ID | residue | amino acid | (SEQ ID | amino acid | (SEQ ID | amino acid | tag (C- |
| NO:) | (N-term) | residues | NO:) | residues | NO:) | residues | term) |
| SEQ: 7 | | Ala-Met-Gly | SEQ: 12 | — | SEQ: 6 | — | — |
| | | Gly | | | | | |
| SEQ: 9 | | Ala-Met- | SEQ: 12 | Gly-Ser-Gly | SEQ: 6 | Lys-Gly | — |
| | | Gly | C-terminal | | | | |

The present invention further relates to an isolated nucleic acid molecule encoding the fusion protein according to the present invention. Especially preferred isolated nucleic acid molecules according to the present invention comprise a nucleic acid sequence according to SEQ ID NO: 8 and 10. The present invention further relates to a vector comprising the nucleic acid molecule according to the present invention. Said vector may provide for the constitutive or inducible expression of said fusion protein according to the present invention.

The invention also relates to a method for obtaining said fusion proteins from a micro-organism, such as a genetically modified suitable host cell which expresses said fusion proteins. Said host cell may be a micro-organism such as bacteria or yeast or an animal cell as e.g. a mammalian cell, in particular a human cell. In one embodiment of the present invention the host cell is an Escherichia coli cell. The host may be selected due to mere biotechnological reasons, e.g. yield, solubility, costs, etc. but may be also selected from a medical point of view, e.g. a non-pathological bacteria or yeast or human cells. Another aspect of the present invention is related to a method for genetically transforming a suitable host cell in order to obtain the expression of the fusion proteins according to the present invention, wherein the host cell is genetically modified by the introduction of a genetic material encoding said fusion proteins into the host cell and obtain their translation and expression by genetic engineering methods well known by the man skilled in the art.

The fusion proteins may be obtained from a micro-organism, such as a genetically modified suitable host cell which expresses said fusion proteins. Said host cell may be a micro-organism such as bacteria or yeast or fungi or an animal cell as e.g. a mammalian cell, in particular a human cell. In one embodiment of the present invention the yeast cell is a Pichia pastoris cell. The host may be selected due to mere biotechnological reasons, e.g. yield, solubility, costs, etc. but may be also selected from a medical point of view, e.g. a non-pathological bacteria or yeast, human cells.

In a further aspect the present invention relates to a composition, preferably a pharmaceutical composition, comprising a fusion protein according to the present invention and/or a host transformed with a nucleic acid molecule or a vector comprising a nucleotide sequence encoding a fusion protein according to the present invention.

The present invention also relates to fusion protein according to the present invention and/or a host transformed with a nucleic acid comprising a nucleotide sequence encoding a fusion protein according to the present invention for use as a medicament. In a further aspect the present invention relates to the use of a fusion protein according to the present invention and/or a host transformed with a vector comprising a nucleic acid molecule comprising a nucleotide sequence encoding a fusion protein according to the present invention in the manufacture of a medicament for the treatment and/or prevention of a disorder, disease or condition associated with Gram-positive bacteria, preferably staphylococcal bacteria, more preferably Staphylococcus aureus. In particular the treatment and/or prevention of the disorder, disease or condition may be caused by Gram-positive bacteria of bacterial groups, families, genera or species comprising strains pathogenic for humans or animals like Staphylococcus aureus.

In another specific embodiment of the present invention the disorder, disease or condition is caused by Staphylococcus aureus, in particular infections of the skin, like pyoderma, particularly folliculitis, furuncle, carbuncle, abscesses of the sweat glands and pemphigus, and like scaled skin syndrome. The scaled skin syndrome can appear in three clinical pictures: dermatitis exfoliativa, impetigo bullosa and scarlatiniform erythroderma. Moreover the disorder, disease or condition caused by Staphylococcus aureus is Staphylococcus pneumonia, hospitalism, in particular surgical wound infections, mastitis puerperalis and enterokolitis, and food poisonings.

The present invention further relates to a medicament comprising a fusion protein according to the present invention and/or a host transformed with a nucleic acid comprising a nucleotide sequence encoding a fusion protein according to the present invention.

In a further aspect the present invention relates to a method of treating a disorder, disease or condition in a subject in need of treatment and/or prevention, which method comprises administering to said subject an effective amount of a fusion protein according to the present invention and/or an effective amount of a host transformed with a nucleic acid comprising a nucleotide sequence encoding a fusion protein according to the present invention or a composition according to the present invention. The subject may be a human or an animal.

In particular said method of treatment may be for the treatment and/or prevention of infections of the skin, of soft tissues, the respiratory system, the lung, the digestive tract, the eye, the ear, the teeth, the nasopharynx, the mouth, the bones, the vagina, of wounds of bacteremia and/or endocarditis caused by staphylococcal bacteria, in particular by the staphylococcal bacteria as listed above.

The dosage and route of administration used in a method of treatment or prophylaxis according to the present invention depends on the specific disease/site of infection to be treated. The route of administration may be for example oral, topical, nasopharyngeal, parenteral, intravenous, rectal or any other route of administration.

For application of a fusion protein according to the present invention and/or an effective amount of a host transformed with a nucleic acid comprising a nucleotide sequence encoding a fusion protein according to the present invention or a composition according to the present invention to a site of infection or site endangered to be infected a formulation may be used that protects the active compounds from environmental influences such as proteases, oxidation, immune response etc., until it reaches the site of infection. Therefore, the formulation may be capsule, dragee, pill, powder, suppository, emulsion, suspension, gel, lotion, cream, salve, injectable solution, syrup, spray, inhalant or any other medical reasonable galenic formulation. Preferably, the galenic formulation may comprise suitable carriers, stabilizers, flavourings, buffers or other suitable reagents. For example, for topical application the formulation may be a lotion, cream, gel, salve or plaster, for nasopharyngeal application the formulation may be saline solution to be applied via a spray to the nose. For oral administration in case of the treatment and/or prevention of a specific infection site e.g. in the intestine, it can be necessary to protect a fusion protein according to the present invention from the harsh digestive environment of the gastrointestinal tract until the site of infection is reached. Thus, bacteria as carrier, which survive the initial steps of digestion in the stomach and which secret later on a fusion protein according to the present invention into the intestinal environment can be used.

Preferably, a fusion protein according to the present invention is used for medical treatment, if the infection to be treated or prevented is caused by multiresistant bacterial strains, in particular by strains resistant against one or more of the following antibiotics: streptomycin, tetracycline, cephalothin, gentamicin, cefotaxime, cephalosporin, ceftazidime or imipenem. Furthermore, a fusion protein according to the present invention can be used in methods of treatment by administering it in combination with or in addition to conventional antibacterial agents, such as antibiotics, lantibiotics, bacteriocins or endolysins.

The present invention also relates to a pharmaceutical pack comprising one or more compartments, wherein at least one compartment comprises one or more fusion protein according to the present invention and/or one or more hosts transformed with a nucleic acid comprising a nucleotide sequence encoding a fusion protein according to the present invention or a composition according to the present invention.

In another aspect the present invention relates to a process of preparation of a pharmaceutical composition, said process comprising admixing one or more fusion protein according to the present invention and/or one or more hosts transformed with a nucleic acid comprising a nucleotide sequence encoding a fusion protein according to the present invention with a pharmaceutically acceptable diluent, excipient or carrier.

In an even further aspect the composition according to the present invention is a cosmetic composition. Several bacterial species can cause irritations on environmentally exposed surfaces of the patient's body such as the skin. In order to prevent such irritations or in order to eliminate minor manifestations of said bacterial pathogens, special cosmetic preparations may be employed, which comprise sufficient amounts of the fusion protein according to the present invention in order to degrade already existing or freshly settling pathogenic Gram-positive, preferably staphylococcal bacteria, more preferably Staphylococcus aureus.

In a further aspect the present invention relates to fusion protein according to the present invention for use as diagnostic means in medicinal, food or feed or environmental diagnostics, in particular as a diagnostic means for the diagnostic of bacteria infection caused in particular by Gram-positive bacteria, preferably staphylococcal bacteria, more preferably Staphylococcus aureus. In this respect the fusion protein according to the present invention may be used as a tool to specifically degrade pathogenic bacteria, in particular Gram-positive pathogenic bacteria, preferably staphylococcal bacteria, more preferably Staphylococcus aureus. The degradation of the bacterial cells by the fusion protein according to the present invention can be supported by the addition of detergents like Triton X-100 or other additives which weaken the bacterial cell envelope like polymyxin B. Specific cell degradation is needed as an initial step for subsequent specific detection of bacteria using nucleic acid based methods like PCR, nucleic acid hybridization or NASBA (Nucleic Acid Sequence Based Amplification), immunological methods like IMS, immunofluorescence or ELISA techniques, or other methods relying on the cellular content of the bacterial cells like enzymatic assays using proteins specific for distinct bacterial groups or species (e.g. β-galactosidase for enterobacteria, coagulase for coagulase positive strains).

In a further aspect the present invention relates to the use of the fusion protein according to the present invention for the treatment or prevention of Gram-positive bacterial, preferably staphylococcal contamination of foodstuff, of food processing equipment, of food processing plants, of feed for livestock animals, of surfaces coming into contact with foodstuff such as shelves and food deposit areas and in all other situations, where pathogenic, facultative pathogenic or other undesirable bacteria can potentially infest food material, of medical devices and of all kind of surfaces in hospitals and surgeries.

In particular, a fusion protein of the present invention may be used prophylactically as sanitizing agent. Said sanitizing agent may be used before or after surgery, or for example during hemodialysis. Moreover, premature infants and immunocompromised persons, or those subjects with need for prosthetic devices may be treated with a fusion protein according to the present invention. Said treatment may be either prophylactically or during acute infection. In the same context, nosocomial infections, especially by antibiotic resistant strains like Methicillin-resistant Staphylococcus aureus (MRSA), in particular MRSA from horse, pig and poultry, Methicillin-sensitive Staphylococcus aureus and Oxacillin-resistant Staphylococcus aureus (ORSA) species may be treated prophylactically or during acute phase with a fusion protein of the present invention. Therefore, a fusion protein according to the present invention may be used as a disinfectant also in combination with other ingredients useful in a disinfecting solution like detergents, tensids, solvents, antibiotics, lantibiotics, or bacteriocins.

In a preferred embodiment a fusion protein according to the present invention can be used in methods of treatments by administering it in combination with at least one of the following antibiotics: β-lactams, aminoglycosides, fluoroquinolones, macrolides, novobiocin, rifampicin, oxazolidinones, fusidic acid, mupirocin, pleuromutilins, daptomycin, vancomycin, tetracyclines, sulfonamides, chloramphenicol, trimetoprim, fosfomycin, cycloserine and polymyxin.

In another preferred embodiment a fusion protein according to the present invention can be used in methods of eliminating, reducing or preventing bacterial biofilms of Staphylococcus aureus by administering it in combination with at least one of the following antibiotics: β-lactams, aminoglycosides, fluoroquinolones, macrolides, novobiocin, rifampicin, oxazolidinones, fusidic acid, mupirocin, pleuromutilins, daptomycin, vancomycin, tetracyclines, sulfonamides, chloramphenicol, trimetoprim, fosfomycin and cycloserine.

For the use of the fusion protein according to the present invention for eliminating, reducing or preventing bacterial biofilms as a disinfectant e.g. in hospital, dental surgery, veterinary, kitchen or bathroom, the fusion protein can be prepared in a composition in form of e.g. a fluid, a powder, a gel, or an ingredient of a wet wipe or a disinfection sheet product. Said composition may additionally comprise suitable carrier, additives, diluting agents and/or excipients for its respective use and form, respectively, —but also agents that support the antimicrobial activity like EDTA or agents enhance the antimicrobial activity of the fusion proteins. The fusion protein may also be used with common disinfectant agents like, Alcohols, Aldehydes, Oxidizing agents, Phenolics, Quaternary ammonium compounds or UV-light. For disinfecting for example surfaces, objects and/or devices the fusion protein can be applied on said surfaces, objects and/or devices. The application may occur for instance by wetting the disinfecting composition with any means such as a cloth or rag, by spraying, pouring. The fusion proteins may be used in varying concentration depending on the respective application and the “reaction time” intended to obtain full antimicrobial activity.

In a further aspect the present invention relates to the use of the fusion protein according to the present invention as a food additive or feed additive for livestock animals.

Further scope of applicability of the present invention will become apparent from the detailed description given hereinafter, however, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

The following examples explain the present invention but are not considered to be limiting. Unless indicated differently, molecular biological standard methods were used, as e.g., described by Sambrock et al., 1989, Molecular Cloning: A Laboratory Manual, 2nd edition, Cold Spring Harbor Laboratory Press, Cold Spring Harbor, N.Y.

### Example 1: Cloning of the Fusion Proteins of Staphylococcus aureus Phage 2638A

Ply2638A having an amino acid sequence according to SEQ ID NO: 1 is a modular endolysin of 486 amino acid residues originating from Staphylococcus aureus phage 2638A with putative N-terminal peptidase/amidase domains and a C-terminal peptidoglycan binding domain.

Codon optimized and synthesized gene of Ply2638A was used as a template in standard PCR reaction with KAPAHiFi™ polymerase (PEQLAB, Erlangen, Germany) using the following PCR parameters and the primers are presented in the table below:

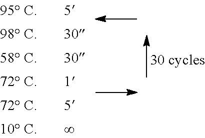

To extend the 5′- or 3′-end of Ply2638A with a fragment encoding the polycationic 9-mer peptide Lys-Arg-Lys-Lys-Arg-Lys-Lys-Arg-Lys (SEQ ID NO: 15) primers presented in table 5 were used for PCR. Ply2638A-PK additionally carries a Gly-Ser-Gly spacer between the Ply2638A and the polycationic peptide.

TABLE 5
Primer sequences used for the cloning of the nucleic acids encoding the fusion
proteins PK-Ply2638A and Ply2638A-PK

| Primer | SEQ ID NO: | Nucleic acid sequence | Experiment |
|---|---|---|---|
| Ply2638A | SEQ ID NO: 35 | cat gcc atg ggc ctg acc gca aft gat | Amplification of the |
| forward | | tac ctg acc | Ply2638A gene |
| Ply2638A | SEQ ID NO: 36 | ccg ctc gag tca tca ttt gat ttc acc | Amplification of the |
| reverse | | cca cag ttt gcc c | Ply2638A gene |
| PK-Ply2638A | SEQ ID NO: 37 | cat gcc atg ggc aag cgt aag aaa cgc | Extension of the |
| forward | | aaa aaa cgc aaa ctg acc gca aft gat | Ply2638A gene |
| | | tac ctg acc | |
| PK-Ply2638A | SEQ ID NO: 38 | ccg ctc gag tca tca ttt gat ttc acc | Extension of the |
| reverse | | cca cag ttt gcc c | Ply2638A gene |
| Ply2638A-PK | SEQ ID NO: 39 | cat gcc atg ggc ctg acc gca aft gat | Extension of the |
| forward | | tac ctg acc | Ply2638A gene |
| Ply2638A-PK | SEQ ID NO: 40 | ccg ctc gag tca tca ttt gcg ttt ttt gcg | Extension of the |
| reverse | | ttt ctt acg ctt tcc cga ccc ttt gat ttc | Ply2638A gene |
| | | acc cca cag ttt gc | |

Both extended fragments were digested with NcoI and XhoI and ligated in a linearized pET32b expression vector (Merck KGaA, Darmstadt, Germany) by following standard cloning protocols. By this way a fusion protein with thioredoxin, S-tag, His-tag and thrombin/enterokinase restriction sites (all comprised in the pET32b vector) was generated.

### Example 2: Purification of the Fusion Proteins of Staphylococcus aureus Phage 2638A

Recombinant expression of proteins is performed in exponentially growing E. coli T7 Express lysY/Iq cells (New England Biolabs, Frankfurt.a.M., Germany) after induction with 0.5 mM IPTG (isopropylthiogalactoside) at 37° C. for a period of 4 hours. Proteins were pre-purified by Ni²⁺ affinity chromatography (Äkta EXPLORER, GE Healthcare) using the His-tag, encoded by the pET32b expression vector. The Ni2+ affinity chromatography is performed in 4 subsequent steps, all at room temperature:

1. Equilibration of the HiTrap IMAC FF 5 ml column (GE Healthcare) with 5 column volumes of Washing Buffer (20 mM imidazole, 1M NaCl and 20 mM HEPES; pH 7.4) at a flow rate of 5 ml/min.

2. Loading of the total lysate (with wanted endolysin/artilysin) on the HiTrap IMAC FF column at a flow rate of 5 ml/min.

3. Washing of the column with 15 column volumes of Washing Buffer at a flow rate of 5 ml/min.

4. Elution of bound protein is done with a 4 column volume gradient to 100% of Elution Buffer (500 mM imidazole, 0.5 M NaCl and 20 mM HEPES; pH 7.4) at a flow rate of 5 ml/min

After pre-purification the fused protein is digested overnight with recombinant enterokinase at room temperature. Ni²⁺ affinity chromatography is performed again, with repeating step 1-3 of the protocol with the target protein in the flowthrough collected in step 3 (His-tagged thioredoxin bound to the column).

### Example 3: Antimicrobial Activity of the Fusion Protein of Ply2638A with Polycationic Stretches on the N-Terminus and C-Terminus

Staphylococcus aureus SP8, Staphylococcus aureus SP10, Staphylococcus aureus SP17, Staphylococcus aureus KS13, Staphylococcus aureus S53 (MRSA), Staphylococcus aureus AV4 (MRSA), Staphylococcus aureus 796N (MRSA) cells (Katholieke Universiteit Leuven, Belgium) as well as Staphylococcus aureus DSMZ 346 and Staphylococcus intermedius DSMZ 20373 (DSMZ, Braunschweig, Germany) were used as test strains.

Overnight cultures were diluted 10-fold in fresh LB medium and grown to OD600=0.6. The culture was spun down and diluted 10-fold in dilution buffer (10 mM HEPES, pH 7.4). Bacteria were incubated at room temperature with each 10 μg fusion protein at a final concentration of 100 μg/ml in buffer (20 mM HEPES 0.5 M NaCl; pH 7.4). After 1 hour cell dilution series were made in PBS and plated on LB. Additionally, a negative control was plated using buffer (20 mM HEPES 0.5 M NaCl; pH 7.4). The residual colonies were counted after an overnight incubation at 37° C. Based on the counted cell numbers the antibacterial activity as logarithmic units (=log₁₀ N₀/Nᵢ with N₀=number of untreated cells and Nᵢ=number of treated cells) was calculated. All samples were replicated at least in four fold.

TABLE 6
Antimicrobial activity of the Ply2638 derivative modified with
cationic peptide stretch against Staphylococcal bacteria

| | PK-Ply2638A | Ply2638A-PK | Ply2638A | Ply2638A-PK | Ply2638A |
|---|---|---|---|---|---|
| S. aureus SP8 | + | +++ | − | 5 | − |
| S. aureus SP10 | + | +++ | − | >4 | − |
| S. aureus SP17 | + | +++ | − | 3 | 0.5 |
| S. aureus S53 (MRSDA) | n.d. | ++ | − | 3 | − |
| S. aureus AV4 (MRSDA) | n.d. | +++ | − | 4 | − |
| S. aureus 796N (MRSDA) | n.d. | +++ | − | 4 | − |
| S. aureus KS13 | n.d. | ++ | − | 3 | n.d. |
| S. aureus DSMZ 346 | − | ++ | − | 2 | − |
| S. intermedius DSMZ 20373 | n.d. | − | − | − | − |
| Staphylococcus aureus ATCC | + | ++ | n.d | 2 | n.d. |
| 6538 | | | | | |

Abbreviations:
−: no activity measured;
+: 0.5-1 log;
++: 2-3 log;
+++: 4 or more logs;
n.d.: not determined;
Reduction of cell numbers is displayed in logarithmic units

### Example 4: Cloning, Expression and Purification of Ply2638A Modified with Various Peptide Stretches on the N-Terminus or the C-Terminus

The endolysin Ply2638A is encoded by the nucleic acid sequence according to SEQ ID NO:2. The nucleic acid sequence according to SEQ ID NO:2 was synthetically produced with a BamH I (5′-GGA TCC-3′) restriction site at the 5′-end of the nucleic acid sequence and an Xho I (5′-CTC GAG-3′) restriction site at the 3′-end of the nucleic acid sequence.

The following peptide stretches were used for the generation of fusion proteins with Ply2638A: Psuedin 1 (SEQ ID NO:41), LL-37 (SEQ ID NO:42), Indolicidin (SEQ ID NO:43), Magainin 44 (SEQ ID NO:44), Pleurocidin (SEQ ID NO:45), Cecropin A (A. aegypti) (SEQ ID NO:46), Buforin II (SEQ ID NO:47), Sarcotoxin IA (SEQ ID NO:48), SMAP-29 (SEQ ID NO:49), OR-7 (SEQ ID NO:50).

The nucleic acid molecules encoding the respective peptide stretches were synthetically produced with a Nco I restriction site plus two additional nucleotides (5′-CCA TGG GC-3′) at the 5′-end of the nucleic acid molecule and a BamH I (5′-GGA TCC-3′) restriction site at the 3′-end of the nucleic acid molecule.

Fusion proteins were constructed by linking at least two nucleic acid sequences using standard cloning techniques as described e.g. by Sambrook et al. 2001, Molecular Cloning: A Laboratory Manual. Therefore the nucleic acid molecules encoding the peptide stretches were cleaved in a digest with the respective restriction enzymes Nco I and BamH I. Subsequently the cleaved nucleic acids encoding the peptide stretches were ligated into the pET32 b expression vector (Novagen, Darmstadt, Germany), which was also cleaved in a digest with the respective restriction enzymes Nco I and BamH I as described above. The modification of the pET32b expression vector refers to the deletion of the sequence encoding a S-tag and the central His-tag.

Subsequently, the nucleic acid molecule encoding the enzyme Ply2638A was cleaved in a digest with the restriction enzyme BamH I and Xho I and ligated into the modified pET32 b expression vector.

The sequence of the endolysin-peptide-fusions was controlled via DNA-sequencing and correct clones were transformed into E. coli BL21(DE3) (Novagen, Darmstadt, Germany) for protein expression.

Recombinant expression of the fusion proteins was performed in E. coli BL21 (DE3) cells (Novagen, Darmstadt, Germany). The cells were growing until an optical density of OD600 nm of 0.5-0.8 was reached. Then the expression of the fusion protein was induced with 1 mM IPTG (isopropylthiogalactoside) and the expression was performed at 37° C. for a period of 4 hours.

E. coli BL21 cells were harvested by centrifugation for 20 min at 6000 g and disrupted via sonication on ice. Soluble and insoluble fraction of the E. coli crude extract were separated by centrifugation (Sorvall, SS34, 30 min, 15 000 rpm). All proteins were purified by Ni2+ affinity chromatography (Akta FPLC, GE Healthcare) using the C-terminal 6×His-tag, encoded by the modified pET32b vector (S-tag and central His-tag deleted), which fuses thioredoxin on the N-terminus of the proteins of interest. The vector also contains an enterokinase cleavage site (DDDDK) right before the protein of interest. This site allows the proteolytic cleavage between thioredoxin and the protein of interest, which can purified via the remaining C-terminal His-tag. Expressed fusion proteins were not toxic to the host resulting in high yields of produced protein. For antimicrobial function of the fusion protein it was necessary to remove the thioredoxin by proteolytic cleavage. Therefore the fusion protein was cleaved with 2-4 units/mg recombinant enterokinase (Novagen, Darmstadt, Germany) according to the manufactures instructions to remove the thioredoxin following the protocol provided by the manufacturer. After enterokinase cleavage the fusion protein was purified via His-tag purification as described below.

The Ni²⁺ affinity chromatography is performed in 4 subsequent steps, all at room temperature:
1. Equilibration of the Histrap FF 5 ml column (GE Healthcare) with up to 10 column volumes of Washing Buffer (20 mM imidazole, 1 M NaCl and 20 mM Hepes on pH 7.4) at a flow rate of 3-5 ml/min.
2. Loading of the total lysate (with wanted fusion protein) on the Histrap FF 5 ml column at a flow rate of 3-5 ml/min.
3. Washing of the column with up to 10 column volumes of Washing Buffer to remove unbound sample followed by a second washing step with 10% Elution buffer (500 mM imidazole, 0.5 M NaCl and 20 mM Hepes on pH 7.4) at a flow rate of 3-5 ml/min.
4. Elution of bounded fusion proteins from the column with a linear gradient of 4 column volumes of Elution Buffer (500 mM imidazole, 0.5 M NaCl and 20 mM Hepes on pH 7.4) to 100% at a flow rate of 3-5 ml/min.

Purified stock solutions of fusion proteins in Elution Buffer (20 mM Hepes pH 7.4; 0.5 M NaCl; 500 mM imidazole) were at least 90% pure as determined visually on SDS-PAGE gels.

### Example 5: Antimicrobial Activity of Ply2638A Modified with Various Peptide Stretches on the N-Terminus or the C-Terminus

### Plating Assay:

Exponentially growing cells of Staphylococci were taken (1 ml) harvested and resuspended in 10 mM HEPES (pH 7.4) and diluted 1:10 in 10 mM HEPES (pH7.4). Fusion proteins were diluted in 20 mM HEPES, 0.5 M NaCl (pH7.4) and incubated with the bacteria for 120 minutes at room temperature. Final concentration of the protein was about 100 μg/ml. After incubation serial dilutions (1:10) were made and bacteria were plated on appropriated agar plates. The residual colonies were counted after an overnight incubation at 37° C. Based on the counted cell numbers the antibacterial activity as logarithmic units (=log 10 N0/Ni with N0=number of untreated cells and Ni=number of treated cells) was calculated (Table 4). All samples were replicated at least in four fold.

### Lysis Test:

Staphylococcal cells of were grown in BHI medium until and optical density at 600 nm of 0.7-1 was reached indicating exponential growth. Cells were harvested by centrifugation and resuspended in 20 mM HEPES (pH 7.4), 250 mM NaCl. Cells were resuspended at an optical density at 600 nm of 1.0 and incubated with fusion proteins. Activity was measured spectrophotometrically at 600 nm.

### Dot Assay:

The dot assay was used test the antimicrobial activity of these fusion proteins against Staphylococcus aureus. Therefore, 200 μl overnight culture of Staphylococcus aureus, were mixed with 3 ml Top-Agar (LB with 0.75% Agar) plated and incubated until a solid agar layer is formed. Then 5 μl fusion protein solutions (ca. 500-800 μg/ml) were dotted. After overnight incubation under appropriate conditions formation of halos has to be analyzed.

### Results:

Six strains of Staphylococcus aureus were tested for their susceptibility to unmodified Ply2638A and Ply2638A-PK. Furthermore Ply2638A-PK was tested on 3 MRSA strains and Staphylococcus intermedius. Ply2638A shows no or only minor reduction in cell numbers compared to negative control without protein. Contrary to the endolysin the Artilysin shows good activity in plating assay using standard protocol (see above) with exception of Staphylococcus intermedius which was lysed neither by the endolysin nor by the Artilysin; see Table 6 above.

Table 7 below shows the results of the fusion proteins having the peptide stretch on the N-terminus of Ply2638A.

Table 8 below shows the results of the fusion proteins having the peptide stretch on the C-terminus of Ply2638A.

TABLE 7

| | LL-37- | SMAP-29- | Cecropin A- | Sarcotoxin IA- | OR-7- | Indolicidin- | Pseudin1- | Magainin- | Pleurocidin- | BuforinII- |
|---|---|---|---|---|---|---|---|---|---|---|
| | Ply2638A | Ply2638A | Ply2638A | Ply2638A | Ply2638A | Ply2638A | Ply2638A | Ply2638A | Ply2638A | Ply2638A |
| S. aureus SP8 | + | ++ | − | − | + | + | − | + | + | − |
| S. aureus SP10 | + | + | + | − | − | + | − | + | − | − |
| S. aureus SP17 | − | + | + | − | + | + | − | + | + | + |
| S. aureus S53 | + | − | + | + | − | + | + | + | − | + |
| S. aureus AV4 | − | + | − | − | − | + | + | + | − | − |
| S. aureus 796N | + | + | − | − | − | + | − | + | + | − |
| S. aureus KS13 | + | + | − | − | + | + | − | − | + | + |
| S. aureus | − | + | − | − | + | + | − | − | − | − |
| DSMZ 346 | | | | | | | | | | |
| S. intermedius | − | − | − | − | − | − | − | − | − | − |
| DSMZ 20373 | | | | | | | | | | |

TABLE 8

| | Ply2638A- | Ply2638A- | Ply2638A- | Ply2638A- | Ply2638A- | Ply2638A- | Ply2638A- | Ply2638A- | Ply2638A- | Ply2638A- |
|---|---|---|---|---|---|---|---|---|---|---|
| | LL-37 | SMAP-29 | Cecropin A | Sarcotoxin IA | OR-7 | Indolicidin | Pseudin1 | Magainin | Pleurocidin | BuforinII |
| S. aureus SP8 | ++ | ++ | ++ | +++ | +++ | +++ | +++ | +++ | +++ | ++ |
| S. aureus SP10 | ++ | +++ | ++ | +++ | ++ | +++ | ++ | + | ++ | ++ |
| S. aureus SP17 | ++ | +++ | ++ | +++ | + | +++ | +++ | +++ | ++ | + |
| S. aureus S53 | +++ | ++ | + | +++ | ++ | ++ | + | ++ | +++ | ++ |
| S. aureus AV4 | + | +++ | ++ | +++ | + | +++ | +++ | +++ | ++ | ++ |
| S. aureus 796N | ++ | + | ++ | +++ | ++ | ++ | ++ | ++ | ++ | + |
| S. aureus KS13 | ++ | +++ | ++ | ++ | + | ++ | +++ | ++ | +++ | + |
| S. aureus DSMZ | ++ | ++ | +++ | ++ | + | +++ | ++ | ++ | +++ | + |
| 346 | | | | | | | | | | |
| S. intermedius | − | − | − | − | − | − | − | − | − | − |
| DSMZ 20373 | | | | | | | | | | |

Abbreviations: −: no activity measured; +: 0.5-1 log; ++: 2-3 log; +++: 4 or more logs; n.d.: not determined

Furthermore, the comparison of photometric lysis tests (using 30 μg/ml protein) showed that fusion proteins contrary to wild type Ply2638A have lytic activity on the test strain Staphylococcus aureus Sp17. With fusion proteins a well-defined clear cut spot appeared within 16 h.

1. A fusion protein comprising a first amino sequence and a second amino acid sequence, the first amino acid sequence comprising a fragment of endolysin Ply2638A that comprises SEQ ID NO: 3, SEQ ID NO: 4 and/or SEQ ID NO: 5, and the second amino acid sequence comprising a sequence heterologous to the first amino acid sequence.

2. (canceled)

3. The fusion protein according to claim 1, wherein the fusion protein comprises SEQ ID NO: 3 or SEQ ID NO: 5.

4. The fusion protein according to claim 1, further comprising a tag, preferably a His6-tag.

5-8. (canceled)

9. An isolated nucleic acid molecule encoding a fusion protein according to claim 1.

10. A vector comprising the nucleic acid molecule according to claim 9.

11. A host cell comprising the nucleic acid molecule according to claim 9.

12. A method of disinfecting a food, feed, cosmetic or environment comprising contacting said food, feed, cosmetic or environment with the fusion protein according to claim 1.

13. A method of treating or preventing staphylococcal bacterial infections in a subject comprising administering to said subject a fusion protein according to claim 1.

14. A method of treating or preventing staphylococcal bacterial contamination of foodstuff, of feed for livestock animals, of food processing equipment, of food processing plants, of surfaces coming into contact with foodstuff, of medical devices, of surfaces in hospitals and surgeries comprising contacting said foodstuff, feed, equipment, plants, surfaces, devices or hospitals with the fusion protein according to claim 1.

15. A pharmaceutical composition comprising the fusion protein according to claim 1.

16. The pharmaceutical composition according to claim 15, further comprising one or more antibiotics.

17. A host cell comprising the vector according to claim 10.

18. The method of claim 12, further comprising contacting with one or more antibiotics.

19. The method of claim 13, further comprising administering one or more antibiotics.

20. The method of claim 14, further comprising contacting with one or more antibiotics.
